(19) United States
(12) Patent Application Publication
(10) Pub. No.: US 2022/0406440 A1
(43) Pub. Date: Dec. 22, 2022

(54) PSYCHOLOGICAL COUNSELING ASSISTIVE SYSTEM EMPLOYING IMMERSIVE VIRTUAL REALITY AND INTERPERSONAL RELATIONSHIP SCULPTURE

(71) Applicant: SUZHOU HB AI TECHNOLOGY RESEARCH&DEVELOPMENT CO., LTD., Suzhou, Jiangsu (CN)

(72) Inventors: Xi WANG, Suzhou, Jiangsu (CN); Yutong GUO, Suzhou, Jiangsu (CN); Erdong WANG, Suzhou, Jiangsu (CN); Jinwen SHEN, Suzhou, Jiangsu (CN)

(21) Appl. No.: 17/776,587

(22) PCT Filed: Sep. 28, 2020
(86) PCT No.: PCT/CN2020/118299

§ 371 Date: May 12, 2022

(30) Foreign Application Priority Data
Nov. 12, 2019 (CN) 201911101569.6

(51) Int. Cl. G16H 20/70

(52) U.S. Cl. CPC G16H 20/70

(57) ABSTRACT

The present invention is directed to a psychological counseling assistive system employing immersive virtual reality (VR) and interpersonal relationship sculpture comprises an interpersonal relationship sculpture construction module and a VR experience module. The interpersonal relationship sculpture construction module comprises an interpersonal relationship sculpture construction basic function module and an interpersonal relationship sculpture construction interaction module. The VR experience module comprises a VR experience function module and a role interaction function module. A VR technique and an interpersonal relationship sculpture technique are combined to allow a visitor to have an immersive experience of an embodiment of a psychophysical field. A character model is used to implement an interpersonal relationship sculpture function that would normally require multiple participants in conventional group psychological intervention. In addition, compared with conventional one-to-one conversation-based intervention, i.e., individual psychological intervention, the system is intuitive and effective, and employs multiple sense channels of the visitor to perform counseling. Using a character model to complete sculpture further facilitates rapid and accurate character interaction.

### FIELD OF THE INVENTION

The invention relates to virtual reality technology, big data, psychological counseling and other related fields, and specifically relates to a virtual reality experience psychological counseling assistive system.

### BACKGROUND

Currently interpersonal issues have become increasingly important issues that have plagued people, and people have paid more and more attention to how to build a good state of mind and interpersonal relationship, how to enhance self-confidence and happiness.

To help people understand their own mental health, common psychological interventions include individual and group psychological interventions. During an individual psychological intervention, a consultant and a visitor are having a one-on-one conversation, that is, there are only two people, a consultant and a visitor; the visitors, for various reasons, reluctant to reveal the pain deep in their hearts are most suitable for this kind of psychological intervention. During a conversation, the consultant often leads the conversation, and sometimes the visitor leads the conversation. In response to related issues, they are having superficial to in-depth conversations. This kind of individual psychological intervention is not good at intuitively action-exploring and expressing visitors' inner interpersonal relationship, thus consultants will spend comparatively more time and effort on visitors' evaluation and intervention.

Group psychological interventions are usually conducted by one to two therapists, and there might be eight to fifteen people with the same or different problems under treatment. People gather together for the treatment, usually once a week, for one and half hours to two hours every time, and the number of treatment may depend on a patient's specific issues and circumstances. During the treatment, group members discuss issues of common concern, observe and analyze the psychological and behavioral responses, emotional experiences and interpersonal relationships of themselves and others, so as to improve their behaviors.

The way of group psychological intervention is more in-depth to visitors than one-on-one consultation, but its disadvantage is that it invites multiple people to participate at the same time, which is also more difficult for group organizers. The more difficult part is the issue of group confidentiality and sense of security.

How to solve the difficulty of inviting people to participate in group psychological interventions, the issue of group sense of security, and at the same time how to overcome the problem of intuition and lack of sense of experience in one-on-one conversation therapy are the urgent problems to be solved for psychological services.

### SUMMARY

The purpose of the present invention is to provide an immersive virtual reality interpersonal relationship sculpture system and a psychological intervention method to overcome the above-mentioned defects in the prior art, which can help visitors focus on their hearts and express their inner worlds more clearly and intuitively; Psychological consultants can at the same time evaluate and intervene visitors based on the character sculpture presented by the visitors in this system; and can record a large number of relevant data, corresponding to psychophysical fields and behaviors, to provide a data collection method for scientific research in psychology.

In order to achieve the above-mentioned purpose of the invention, the present immersive virtual reality interpersonal relationship sculpture psychological counselling assistive system includes an interpersonal relationship sculpture construction module and a virtual reality experience module.

Among them, the interpersonal relationship sculpture construction module is responsible for establishing a virtual reality geographic environment scene, where the geographic environment scene can also be called interpersonal relationship sculpture; the virtual reality interpersonal relationship sculpture includes characters and scenes; the interpersonal relationship sculpture uses interpersonal relationship character models to place and form a sculpture that includes the characters and the environment of the scene.

Wherein, preferably, the interpersonal relationship sculpture construction module comprises an interpersonal relationship sculpture construction basic function module and an interpersonal relationship sculpture construction interaction module.

Furthermore, the interpersonal relationship sculpture construction basic function module comprises a character model library unit and a virtual scene model unit.

Wherein, the interpersonal relationship sculpture construction interaction module comprises a character placement interaction unit and a virtual scene setting interaction unit.

The interpersonal relationship sculpture character model library unit stores a character model for expressing the user's internal important interpersonal relationship; wherein the user's intrinsic important interpersonal relationship is a family relationship, a friend relationship or a colleague relationship; the family relationship is a parent-child relationship, a husband-wife relationship, or sibling relationship.

Furthermore, the character model used to express the important interpersonal relationship is set according to the character image requirements, where the character image requirements include different genders, ages, heights, fat and thin; herein, considering the factors of the psychophysical field and the projection function, preferably the character model can not only express the realistic image of the user's internal interpersonal relationship object, but also can be used by the user to express the object's self-interpreted abstract image.

Preferably, the material of the character model can be set.

The virtual scene model unit stores scene models for users to place character models of themselves and important others, and is responsible for constructing a kind of virtual scene space to ensure visitors' sense of security and enhance the sense of psychological projection and substitution.

The virtual scene model unit includes a time model and a basic space element model.

Specifically, the time model includes one or more types of day, evening, or night;

Furthermore, the time model includes seasons or weather elements; the weather may be heavy rain, storm, or snow; and the season is spring, summer, autumn or winter.

Specifically, the basic space element model includes one or more types of ground, sky, or wall;

Furthermore, the basic space element model includes mountains, rivers, flowers, forests, sun, moon or stars.

Preferably, the sky can be switched according to the mood of the user.

Furthermore, the material of the basic space element model can be set.

Furthermore, the virtual scene model unit includes a lighting unit, a material unit or a post-processing unit.

Wherein, the lighting unit is used to provide lighting design for the entire virtual scene; the material unit is used to implement material settings for the scene model and the character model.

Wherein, the wall material is preferably a beige hard wall during the day or a blue transparent fluorescent material at night.

The post-processing unit is used to adjust and optimize the overall look and feel of the scene, to create a real virtual environment and a virtual environment that is more in line with the user's psychological cognition.

Furthermore, the interpersonal relationship sculpture construction interaction module includes a virtual scene setting interaction unit and a character model placement interaction unit; the interpersonal relationship sculpture construction interaction module is established based on the interpersonal relationship sculpture construction basic function module.

Wherein, the interpersonal relationship sculpture construction interaction module is responsible for the selection and placement of character models of users (including visitors and psychological counselors) and the formation of a virtual reality experience environment.

Wherein, the virtual scene setting interaction unit is used to provide a scene environment in which the character model is placed, including one or more settings of time, space, lighting, and model material.

The character model placement interaction unit assists the user to complete the selection and placement of the character model.

Specifically, when the user selects his own character model, he can select the character model, according to the user's real character and the image of the character that the user imagines to be. That is, the character model selected by the user is the model of the user's real character and/or the character that the user imagines to be; this is to meet the needs of users for multiple experience modes, such as role substitution, meditation, etc.

The character model placement interaction unit assists the user in setting the position of the character model, the distance between the character models, or the orientation of the character model etc.

Furthermore, the character model placement interaction unit comprises a function to highlight the characteristics of the characters, which include rights, status etc.

Wherein, preferably, a 3D perspective is used during the character placement stage.

The character model placement interaction unit includes 9 character model placement function modules, 9 functional modules, followed by the character model generation function module, the character model selection function module, the character model rotation function module, and the character model deletion function module, the character model movement function module, the character operation cancellation function module, the character model color change function module, the character model height adjustment function module and the data recording function module in the interpersonal relationship sculpture.

Wherein, the data recording function module is to record and collect the data of the interpersonal relationship character sculpture.

The character model generation function module is used for the user to select the required character model and generate the selected model in the model placement area.

The character model selection function module is used to select the character model in the model placement area.

The character model rotation function module is used for the user to rotate the model in the area where the model is placed.

The character model deletion function module is used to delete the character model in the model placement area.

The character model operation cancellation function module is used to cancel the pre-placement state of the character model, when the user selects the character model in the character model selection area, but does not want to place the model in the character model placement area.

The character model movement function module is used for the user to modify the position of the character model.

The height adjustment function module of the character model is used to improve the position of the character model in the area where the character model is placed.

The character model color change function module is used to change the color of the character model in the area where the character model is placed; the color of the character is to assist the visitor to project the feeling and cognition of the character through the synesthesia of human perception, so as to help the consultant better understand the character's role in the mind of the visitor through the feeling.

The virtual reality experience module includes a VR experience function module and a character interaction function module.

The VR experience function module is used for visitors to enter the completed interpersonal relationship sculpture for experience.

The role interaction function module helps the user to enter the character models of different target positions in the interpersonal relationship sculpture for role interaction; for preparations for psychological interaction experience; the target position model is a user's own character model, an important person's character model or a mirrored character model.

The role interaction function module assists the user to complete the role interaction through the role interaction controller. The role interaction is to help users get a psychological experience and get a deep understanding of themselves and their important others. The role interaction function module helps users obtain the experience of role exchange, role substitution, mirroring, meditation or time travel etc.

Preferably, the character interaction controller is a handle controller.

Specifically, the role interaction function module assists the user to enter the target character model through the role interaction controller, it comprises the following steps:

(1) The user triggers the role interaction controller;

(2) The role interaction function module tracks the target character model through the role interaction controller, confirms the validity of the target character model, and outputs the target character model information;

(3) The role interaction function module obtains the position and orientation of the target character model, from the target character model information output, and records the position data of the target character model;

(4) The role interaction function module obtains the user's position in the virtual space according to the position of the VR main helmet;

(5) Role interaction: the role interaction function module sets the user's position as the position of the target character model, and the user presses the button on the character interaction controller to enter the target character model perspective;

(6) High degree of viewing angle processing and matching: to achieve the complete coincidence of the contours of the user's body and the target character model;

Wherein, the character interaction controller traces the target character model through ray.

Preferably, the process of the target character model ray tracing is:

(1) The role interaction function module determines the starting position of the search ray, according to the position and direction of the current role interaction controller, and starts the search ray tracing function. The role interaction controller emits a ray to move the ray to the character model of the character to be interacted to select the target character model;

(2) After confirming the validity of the target character model, the role interaction function module loads the target character model information and outputs the target character model information.

Furthermore, the virtual reality experience module provides the handle controller to assist the user in switching perspectives in the virtual reality environment.

Furthermore, the virtual reality experience module provides the handle controller to assist the user in grabbing or moving the character model in the virtual reality environment;

Furthermore, the virtual reality experience module provides the handle controller to assist the user to change the position or orientation of the character model remotely, thereby changing the space of the interpersonal relationship sculpture.

Furthermore, this application provides a psychological intervention method based on the aforementioned immersive virtual reality interpersonal relationship sculpture psychological counseling assistive system, which is characterized in that it includes the following steps:

(1) The user enters the psychological counseling assistive system to set up the virtual scene of the interpersonal relationship sculpture;

(2) The user chooses the character model of himself and others;

(3) The user puts the character model in the virtual scene, and the system generates the user's interpersonal relationship sculpture;

(4) The user puts on the virtual reality device and enters the virtual reality environment;

(5) After the user enters the virtual reality environment, he confirms the scene;

(6) After the user is satisfied with the current interpersonal relationship sculpture, under the guidance of the consultant, through the handle controller, the user chooses to enter the perspective of different character models and experiences it.

Furthermore, in step 5, the scene confirmation includes that the user switches to the perspective view through the handle controller, and observes in the established interpersonal relationship sculpture; if the user is not satisfied with the current interpersonal relationship sculpture, the user can grab or move the position or direction of the character model through the handle controller; or return to step 1 or step 2 to adjust the virtual scene or character model settings.

Furthermore, the above psychological intervention method comprises step 7. After the experience is completed, if the user is satisfied with the experience, the experience is ended; if the user is not satisfied with this experience, he can switch to the perspective view through the handle controller, and use the handle controller to grab and move the position or direction of the character model to change the interpersonal relationship sculpture; The system records the position or direction data of the character model after the change. After that, the user again selects the perspective of different character models through the handle controller, under the guidance of the consultant, to experience again.

Preferably, in step 2, the user selects a mirrored character model, and the mirrored character model is a bystander character model outside the user's interpersonal relationship.

Preferably, the character model selected by the user in step 2 is the real character model of the user or the character model of the character that the user imagines to be.

Preferably, in step 6, the perspectives of different character models that the user chooses to enter include the user's own character model, the other person's character model, or the perspective of the mirrored character model; wherein the other person's character model is the character model of other members in the interpersonal relationship.

Furthermore, the virtual scene setting in step 1 comprises setting of time, space or lighting.

Furthermore, the character model selection in step 2 comprises the selection of the character model's gender, age, height, or the selection of fat or thin character models.

The present invention has the following beneficial effects:

(1) The function of this system that uses character models to complete family sculptures or interpersonal relationship sculptures, that require multiple people to play in traditional group psychological interventions, not only solves the difficulty of inviting participation in the form of group psychological intervention and the problem of group security, but also provides visitors with the same way of expressing and reflecting on interpersonal motivation. Compared with the traditional one-to-one talk therapy, the method of this system is more intuitive and effective, and it can mobilize multiple channels of visitors for consultation.

(2) This product can provide users with an immersive experience, and VR can provide users with a more complete physical experience of the psychophysical field. This system uses virtual reality (VR) technology combined with family sculpture technology to provide visitors with an immersive psychophysical physical experience. Traditional group family therapy can also give users a similar integrity experience, but in comparison, this product is more cost-effective, and can record a large amount of psychophysical field and behavioral related data. This provides a data collection method for scientific research in psychology, and helps researchers build a larger database for further in-depth exploration based on existing clinical psychology theories.

(3) The role interaction function module enables users to enter different target roles to prepare for the psychological interaction experience. The role interaction function module uses the character model to make the user enter the target role, and prepares the user for the psychological experience of empathy, mirroring, role substitution, time and space travel, meditation and other modes. Compared with the traditional psychological intervention method, this does not require a real person to play, which can improve the user's psychological projection and sense of substitution, and help users get a deep experience. In this system, we use the character model to complete the sculpture, another advantage is that it is convenient for quick and accurate character interaction.

(4) It provides a tool (medium) for psychological counseling to help visitors express the state of internal imaging and interpersonal relationships in a pictorial way. According to the visitor's placement process and the final sculpture, the psychologist can evaluate the visitor's internal imaging.

### BRIEF DESCRIPTION OF THE DRAWINGS

FIG. 1 is a flow chart of the system principle of the present invention.

FIG. 2 is a diagram of the system structure of the present invention.

FIG. 3 is an image of a character model in an embodiment of the present invention.

FIG. 4 is a schematic diagram of a virtual scene and a character model constructed in an embodiment of the present invention.

FIG. 5 is a schematic diagram of the placement of character models in an embodiment of the present invention.

FIG. 6 is a schematic diagram of editing a character model in an embodiment of the present invention.

FIG. 7 is a schematic diagram of a virtual reality experience environment in an embodiment of the present invention.

FIG. 8 is a flowchart of specific implementation steps of the virtual reality experience link in an embodiment of the present invention.

FIG. 9 is a schematic diagram of the definition of role interaction in an embodiment of the present invention.

FIG. 10 is a schematic diagram of the process of character tracking in an embodiment of the present invention.

FIG. 11 is a schematic diagram of the flow of role transmission in an embodiment of the present invention.

### DETAILED DESCRIPTION

In order to make it easy to understand the technical means, creative features, objectives and effects achieved by the present invention, the following embodiments are combined with the accompanying drawings to illustrate the present invention in detail.

According to related psychological theories, the concept of human perception of reality is called the psychological field, and the manifestation of the perception is called the physical field. There is no one-to-one correspondence between the two, but human mental activity is a combination of the two. Specifically, the psychological activity of an organism is a field of dynamic interaction between self-behavior environment-geographical environment etc. This system builds a virtual reality “geographical environment” scene, with the language assistance of a psychological counselor, to help the visitor use the self function to project the internal behavior environment into the scene, and then project the interpersonal relationship existing in the internal self into the VR world. Psychological counselors can use this projected interpersonal relationship dynamics to evaluate and intervene in the internal structure and development of the client.

Therefore, the scene needs to meet the following requirements: firstly, the scene cannot be too real and complicated, which may easily lead to failure of projection; secondly, the overall environment of the scene needs to be focused within a safe distance of the visitor, so as to give the visitor a comfortable feeling and facilitate the projection; Finally, the scene does not involve too many motion elements as much as possible (especially the motion with unfixed frequency), which easily affects the stability and security of the visitor, and may also cause the problem of VR dizziness.

In order to establish a virtual “geographical environment” scene, it is necessary to establish the required interpersonal relationship sculpture character library and virtual scene database.

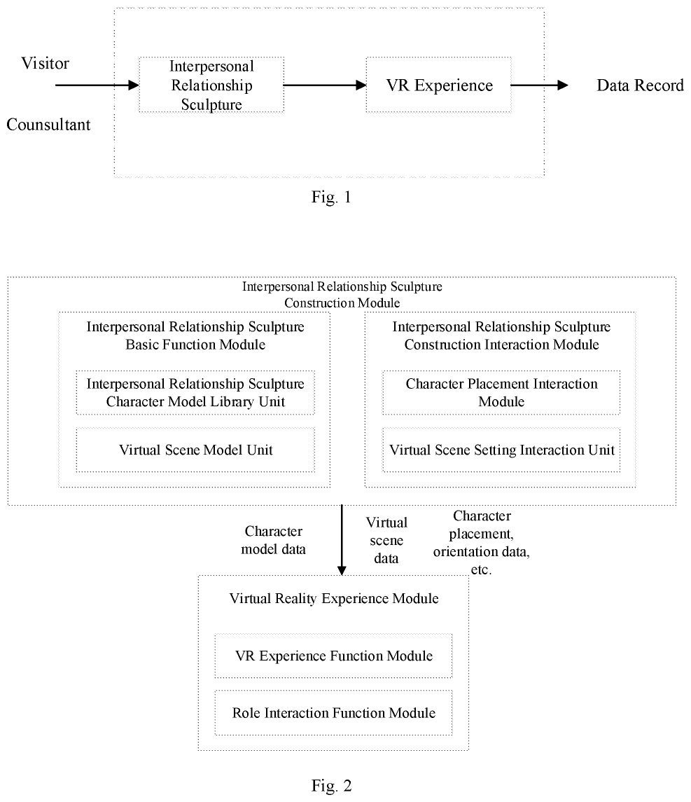

The interpersonal relationship sculpture character library comprises character models used to express the user's internal important interpersonal relationship; among them, the user's internal important interpersonal relationship includes family relationship, friend relationship or colleague relationship; family relationship includes parent-child relationship, husband and wife relationship or sibling relationship; the schematic diagram and structural block diagram of the system are shown in FIG. 1 and FIG. 2.

In this embodiment, family relationships in important interpersonal relationships are selected to establish an immersive virtual reality family sculpture psychological counseling assistive system.

1. Family Sculpture Construction Module

1) Family Sculpture Construction Basic Function Module

a) Character Model Library

The character model library used to express the important task relationship needs to contain all the possibilities of human appearance in the real world, including different gender, age, height, weight, skin color, body shape, hairstyle and clothing; at the same time, taking into account the factors of mind and object field and projection function, the character model should not be too pursued to conform to reality; in this way, the character model we provide cannot only represent the realistic image of the user's internal interpersonal relationship object, but can also be used by the user to express the user's abstract image after self-interpretation has been added.

Here, as one of the preferred schemes, twelve basic character models are designed, namely babies (model no. 1), young females (model no. 2), young males (model no. 3), and adult females (model no. 4), adult males (model no. 5), elderly males (model no. 6), males and females with more toned bodies (models no. 7 and no. 8), males and females with fatter bodies (models no. 9 and no. 10) and burly character models (models no. 11 and no. 12).

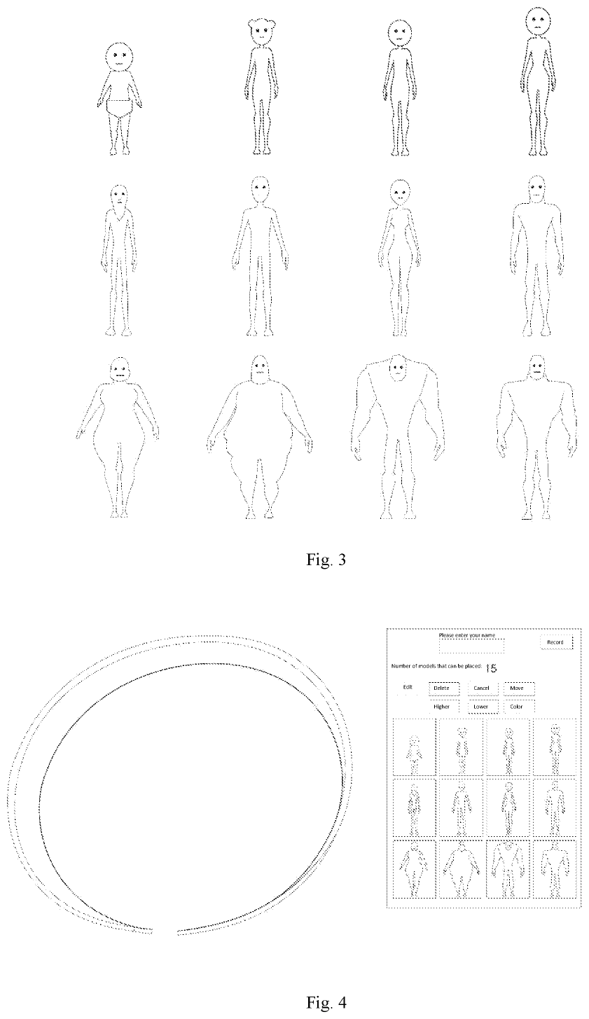

The specific images of these models are shown in FIG. 3.

Taking into account the concept of projection in psychology, the poly style character image is used as the representative of the family sculpture character model. The model is extremely simple, except for simple gender characteristics and tall, short, fat and thin styles, without adding imagery as much as possible, and even hair is not involved. When humans project their own internal structure into the real world seen by the naked eye, the higher the ratio of the elements that can cause the projection and the disturbing image (the image with the inner image clearly conflicting) in the geographical environment, the easier it is to complete the formation of the behavioral environment.

In order to meet the needs of users for the visualization of their own internal characters, as another preferred solution, a set of character models have been recreated according to the stages of human growth. The specific content is shown in the following table:

TABLE 1

| | | | | | | | | | | | | |
|---|---|---|---|---|---|---|---|---|---|---|---|---|
| Body | | Male | Female | Male | Female | Young | Young | Middle- | Middle-aged | Elderly | Elderly | |
| Type | Babies | child | child | teenager | teenager | man | woman | aged man | woman | man | woman | Total |

| | | |
|---|---|---|
| Fat | No need to | 42 |
| Thin | be tall, | character |
| Tall | short, fat | models |
| Short | or thin | |
| | (divided | |
| | into a | |
| | baby model | |
| | lying in a | |
| | cradle and | |
| | a standing | |
| | baby model) | |

The baby character model may be a standing baby model or a baby model in a cradle.

b) Virtual Scene Model Design

After the character model library is established, we also need to construct a virtual geographic environment scene space to provide users with an area or space to place the character models of important others, and construct a space similar to the real world environment to ensure a basic sense of security and enhance the user's psychological projection and sense of substitution; in the psychological sense, this space emphasizes the projection of the user's inner psychological world in the virtual reality space, which means that this space needs to be able to express the user's inner feelings, style, personality characteristics and basic structure and other inner mental states;

The system can also dynamically and automatically change the overall style and detail elements of the scene based on the real-time collection, analysis and feedback of the user's physiological and psychological data, thereby providing users with a realistic experience of projecting the inner world into the outer space.

The virtual environment scene model includes time scene model and basic space model design;

Preferably, here, we build a cylinder with a diameter of 1000 m and a height of 0.2 m as the ground. Any other shape of ground, even non-ground (such as water), can be used; a hollow cylinder with a radius of 10 m, a height of 2.5 m, and a thickness of 0.2 m serves as the wall. A transformable sky ball is set up to simulate the realistic sky environment.

Preferably, the system has designed three time scene models comprising daytime, evening and night; the time scene models can also comprise seasons or weather elements, which more vividly simulate the inner psychological scene of the user.

Specifically, the basic spatial element model comprises ground, sky, and walls.

Preferably, here we consider that the circular ground can simulate an infinitely extending surface effect, giving visitors a feeling of being in an infinite open space. No other imagery is added, so that the visitor can concentrate on his inner space.

Walls are used to separate the inner psychological space and external things, and give visitors a feeling of being protected inside. The first chosen type of wall is an opaque and untouchable separation wall; the second type is a transparent interconnection wall, which gives visitors a feeling of connecting with the outside world.

The sky can be switched according to the mood of the visitor, and it can also help the psychologist to better experience the emotional tone of the visitor's inner world.

The prominent features in each time scene design are different. As a preferred embodiment, the daytime scene uses sunny days, blue sky, white clouds and soft yellowish environment, in order to give people a warm and bright feeling, and give visitors a relaxing and comfortable experience; the evening scene uses the 18:00 pm sky, the sunset and the light blue environment, in order to give people a soft and quiet feeling, and give visitors a relaxing and quiet experience; the night scene uses a visual environment of deep starry sky and dark blue layering, in order to give people a very tense stage effect, and give visitors an experience of deep thinking and exploring inner mysteries. The system can also implement other settings to achieve different purposes. Lighting settings: the system can use multiple light sources arbitrarily. Preferably, the system comprises four types of simulated light sources, namely: directional light source, point light source, spot light source, and sky light source.

The sky light source is used to provide a basic background space lighting effect for the entire scene and use it to illuminate the scene meshes; however, when the sky light is dark, the scene requires other light source designs. Therefore, we control the intensity and direction of the light by placing a directional light source in the scene, which can be changed according to different sky conditions, so that the lighting of the main scene can meet the basic visual needs of visitors. The spot light source and the point light source are used to optimize the evening and night environment; specifically, 1 to 3 spot light sources are placed on the ground, which can increase the intensity of night light to make up for the lack of light in the night sky and optimize the effect of night scenes.

In the preferred embodiment, we use three weak light sources in the evening scene, which can basically cause the shadow of the characters to be lighter. From the perspective of counseling psychology, darkness easily reminds people of negative images such as shadows, pain, and fear, so we try to use a softer and warmer style as much as possible in the design of the evening scene.

As the preferred embodiment, the design style of the late night scene is more like exploring one's inner self in the subconscious deep sea, so a single strong light source is chosen to express the style with greater tension.

Material setting of character model and virtual scene model: the character model and virtual scene model material setting unit is used to generate the material effect of the scene model and the character model.

For the ground material, preferably, there are three different ground materials:

The checkerboard material: the light yellow checkerboard hard floor style is used here. This style is not only convenient for the operator to confirm the position of the character model, but also can give visitors a warm and homelike feeling.

The ocean material: the ocean material is produced by using ocean texture on the ground. In the night scene, this special material will give visitors a feeling of standing on the surface of the deep sea, and the sea at night gives us the feeling of being connected with the inner space.

The lake surface material: the lake surface material is produced by placing the lake surface texture on the ground. In the evening scene, this special material will give visitors a feeling of standing on a peaceful lake. It is similar to the texture of the ocean, but the lake chosen here emphasizes the feeling of warmth and tranquility.

Regarding the wall material, the wall material is divided into two types: one is a beige hard wall during the day; the second is a blue transparent fluorescent material at night. The first is to create a sense of family warmth in line with the ground. The second one is designed in consideration of the deep and broad sense of the evening and night scenes.

Post-processing: This system uses post-processing units to further adjust and optimize the overall look and feel of the scene.

As a preferred embodiment, four post-processing volumes are added to the scene to enhance the visual effects of the scene: including color grading, automatic exposure, lens flare, and depth of field. These post-processing volumes compensate for the defects of the camera lens by simulating human vision to create a more realistic virtual environment. At the same time, we also added atmospheric fog and reflective balls to continue to optimize the scene environment.

2) Family Sculpture Construction Interaction Module

a) Virtual Scene Setting Interaction Module

When placing the character model, it is necessary to set up the family sculpture display area and the character model first, and the family sculpture display area is used to implement the basic environment of the family sculpture. That is, based on the virtual scene model unit mentioned above, it is used to place the user's family sculpture and form a virtual reality experience environment, including setting time, space, lighting and model materials, etc.;

Before setting the virtual scene, the user can set the number of characters who choose to be brought into the virtual world for sculpture experience and their physical height. The setting of the real height during the VR experience can make the user experience more real and the effect is better.

b) Family Sculpture Construction Character Model Placement Interaction Units

Family sculpture means that the relationship between family members is placed using character models to form a complete sculpture. During the placement process, the consultant will inform the visitor of the meaning of the distance between the characters and the orientation: the physical distance between the characters is the inner psychological distance; the orientation of the characters is the relationship or thing that they focus on. For example, if a visitor's father is absent from the visitor for a long time due to work reasons, the visitor and his father's sculpture may be physically far away (5-8 meters away), and the father's orientation may be facing away from the visitor towards the door or outside the wall.

The system is also designed with functions which highlight the characteristics of people with higher rights and status.

The color of the character is to assist the visitor to project the feeling and cognition of the character through the synesthesia of human perception, thereby helping the consultant to understand the character's role in the mind of the visitor at a more perceptual level.

The family sculpture construction character model placement interaction unit is mainly used to place the character model in the family sculpture placement area, and has all the basic functions and requirements for implementing the placement of the family sculpture's character model.

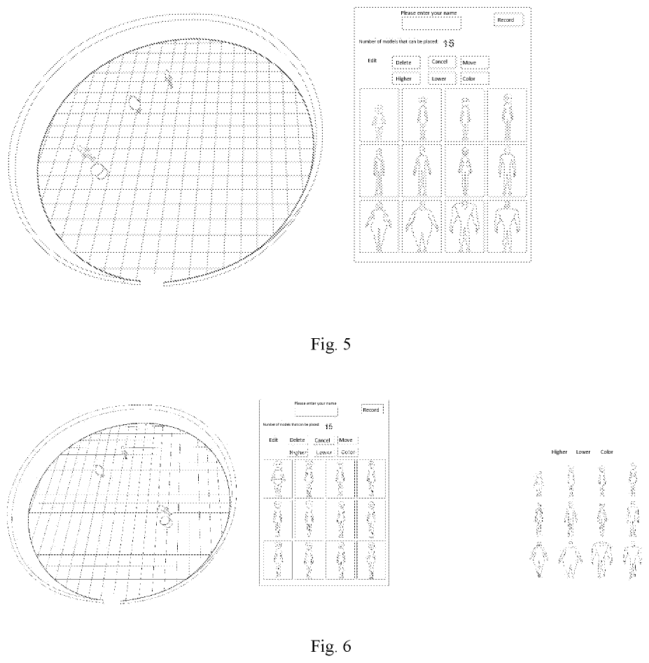

As shown in FIGS. 4 and 5, the number of characters that the visitor chooses to bring into the virtual world for sculpture experience is 15. Preferably, the visitor selects the character model from the 12 character models that appear in the character model selection area, and puts it into the display area on the left.

In order to complete the placement of the user's family sculpture, there are 9 functional modules in the family sculpture, the character generation function module, the model selection functional module, the model rotation functional module, the model deletion functional module, the model moving functional module, and the operation cancellation functional module, color change function module, height adjustment function module, data recording function module. For the convenience of description, the “models” mentioned below are all character models used in the family sculpture.

Among them, the data recording function module is to record and collect the data of the interpersonal relationship sculpture character model. The visitor enters his own user ID, and through the recording function, all data is recorded in a set format. Whenever the user places a character model, the number of characters is displayed minus one in real time. The maximum number of placements is set in the setting module of the starting unit.

The model generation function module is used for the user to select the required character model, and generate the selected model in the model placement area;

Here, the user chooses the character model of himself and important others based on the interpersonal relationship. In order to meet the user's needs for multiple experience modes such as role substitution, mirroring or meditation, it is preferable that when the user selects his own character model, it includes selecting the user's real character model or the character model of the user experience character; the user's real character model corresponds to the user's real character in the interpersonal relationship; the user experience character model is the persona that the user imagines to become in the interpersonal relationship; the user can also choose the mirrored character model, which is the character model of the bystander outside the user's interpersonal relationship.

The model generation function module provides a character model selection area. There are twelve models to choose from. Firstly, the user selects the character model he wants to select, and other models are not selectable. Then, the user selects the character model in the circle on the left side. After the location is determined, the selected model is generated at the selected location. At this time, the state of the selection area of the family sculpture model is restored and can be selected again.

The model selection function module is used to select models in the model placement area.

The model rotation function module is used for the user to rotate the model in the area where the model is placed.

The character model placement interaction unit also provides other character model placement function modules, and the model deletion function module is used to delete the selected character model in the placement model area.

The model operation cancellation function module is used to implement the cancellation of the pre-arrangement state of the character model, when the user selects a character model in the character model selection area, but does not want to place the model in the character model placement area.

The model moving function module is used for users to modify the position of the model.

The model height adjustment function module is used to improve the position of the character model in the area where the character model is placed. In a preferred embodiment, a cylindrical step is generated at the foot of the character model to improve the position of the character model.

The model color change function module is used to change the color of the character model in the character model placement area. Firstly, the user selects the character model whose color needs to be changed in the character model placement area on the left, and selects the color from the colors provided by the color selection unit to change the color of the selected character model. In a preferred embodiment, the color selection unit includes a total of eighteen basic colors. After the color is selected, the module returns to the model placement interface.

The family sculpture construction character model placement interaction module can also provide a separate editing area for character models, which can be used to edit and set up the selected character models for height, weight, color, and material; as shown in FIG. 6.

In the stage of character model placement, a 3d perspective, preferably an oblique forty-five degree top-down perspective, is being used. This perspective has three main advantages: 1. This perspective can overlook the scene of the virtual reality experience, and the sculpture in the scene can be modified in real time through the UI integrated in the perspective. 2. This perspective is commonly known as God's perspective, which allows users to have a full sense of control over the overall position of the sculpture during the placement stage. 3. Compared with the overhead perspective of traditional board games, this perspective shows the model and position of the characters more clearly in the form of a 3D picture.

2. Virtual Reality Experience Module

1) VR Experience Function Module

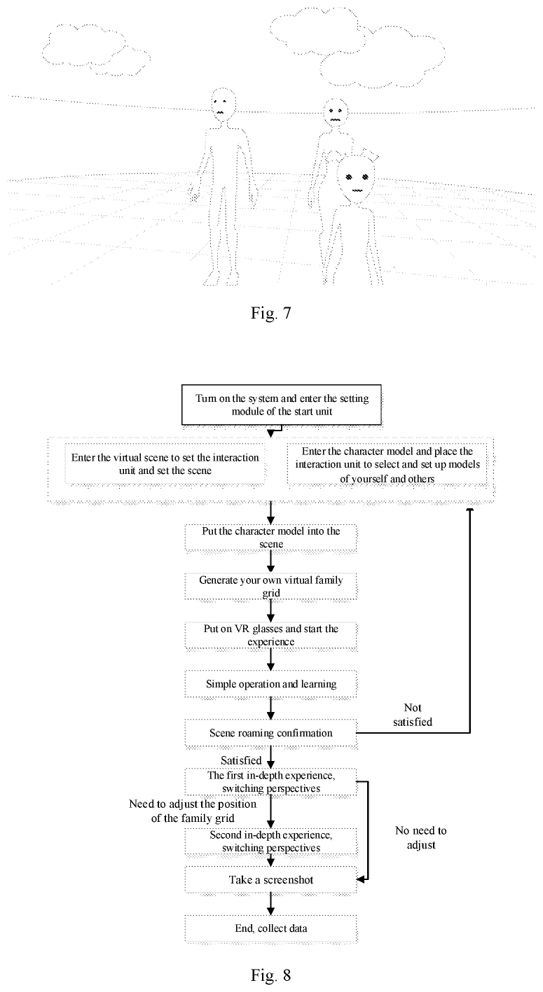

As shown in FIG. 7, after the model is placed, the user switches to the experience module of the system, and brings the virtual reality headset into the just completed family sculpture space to experience the scene roaming experience.

Preferably, the user confirms the scene roaming experience in the virtual reality environment through the handle controller, and specifically uses the handle buttons to switch the perspective, such as switching to the long-range perspective (third-person perspective), observe in the established scene, and confirm whether the user is satisfied with the current family sculpture space, if not satisfied, return to the family sculpture construction interaction module to readjust the characters and scenes;

2) Role Interaction Function Module

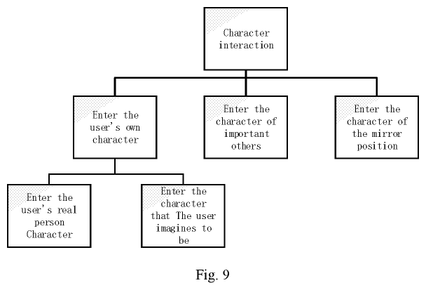

The role interaction function module is used to implement the user's entry into the character model of different target roles, such as entering the user's own role or the role of others, or entering the character model of the mirror role, to prepare for the psychological interaction experience. As shown in FIG. 9, the user's own role can be the user's real role or the user's imaginary role; the mirrored role is the bystander role in the mirrored mode.

Preferably, the role interaction function module assists the user to complete the character interaction through the role interaction controller; according to the guidance of the psychological counselor, the user completes the role interaction function by operating the handle. Role interaction is to gain psychological experience and to gain a deeper understanding of yourself and your important others.

The role interaction function module helps users implement the experience of role exchange, role substitution, mirroring, meditation or time travel.

As a preferred embodiment, after the scene roaming experience is confirmed, if the user is satisfied with the current family sculpture space, he will enter the perspective of different target character models through the handle controller according to the guidance of the consultant for in-depth experience; after completing the in-depth experience, the user confirms if he needs to adjust the family sculpture space for the next experience. If the user needs to adjust the family sculpture space, he can switch to the long-range perspective (that is, the third-person perspective) through the handle button, and use the handle to grab and move the position or direction of the character model from a long distance; Guided by the consultant, through the handle controller, the user can choose to enter the perspective of different target character models.

The role interaction function module assists the user to enter the perspective of different target role character models through the role interaction controller, which specifically includes the following steps:

a) The user triggers the role interaction controller;

Among them, the role interaction controller is preferably a handle controller, and the role interaction is implemented through the buttons on the handle.

b) The role interaction function module tracks the target character model through the role interaction controller, confirms the validity of the target character model, and outputs the target character model information;

Wherein, the target character model refers to the personal character model, the other person character model, or the mirrored character model that the user performs interactive experience. Specifically, the ray tracing target position is used to confirm the validity of the target character model;

The process of ray tracing the target character model is:

i) The role interaction function module determines the starting position of the search ray according to the position and direction of the current role interaction controller, and starts the search ray tracing function. The role interaction controller emits a ray, moves the ray to the character model of the character to be interacted, and selects the target character model;

ii) After confirming the validity of the target character model, the role interaction function module loads the target character model information and outputs the target character model information; validity refers to the way that the target character model transmits the target;

iii) The role interaction function module determines the end position of the search ray, displays the ray effect model after selecting the target character model, and the preferred ray is displayed as a blue column;

c) The role interaction function module obtains the position and orientation of the target character model according to the output target character model information, and records the position data of the target character model;

d) The role interaction function module obtains the user's position in the virtual space through the position of the hardware device (VR main helmet);

e) Role interaction: The role interaction function module sets the position of the user to the position of the target character model, and the user presses the handle button to enter the perspective of the target character model;

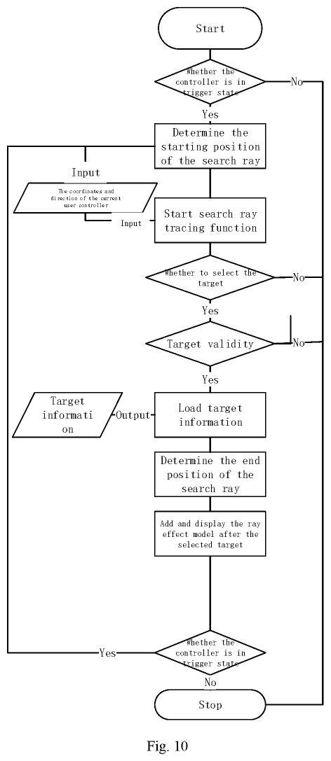

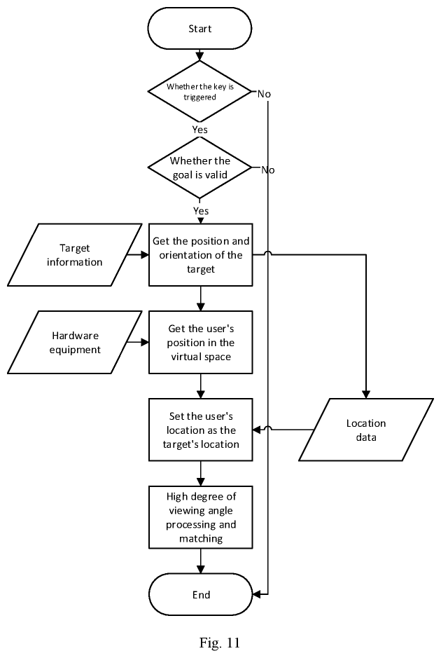

f) High degree of viewing angle processing and matching: it makes the user's body and the outline of the character model completely coincide; the specific tracking flowchart and the character transmission flowchart are shown in FIG. 10 and FIG. 11.

The character model of the user for interactive experience is the real character model of the user or the character model of the character that the user imagines to become; the other person character model is the character model of other members in the interpersonal relationship; the mirrored character model is the bystander character model outside the user's interpersonal relationship.

Taking the role interaction mode of the role exchange model as an example, the empathy mentioned in normal life is different. Role exchange technology can help people quickly withdraw from their own roles and enter another person's world, thereby helping visitors get to know their important others deeply. However, in a psychodrama group, there are many ways to exchange roles, but they all need the auxiliary characters to move their positions to match the protagonist's entry into the opponent's role. This type of movement can sometimes cause confusion. In this system, another advantage of using character models to complete the sculpture is that it is convenient to perform role exchanges quickly and accurately, and to conduct dialogue with the assistance of the consultant.

The user can face the role model of the other party who wants to exchange, and press the relevant button on the handle without letting go. At this time, a ray will be emitted from the front end of the handle, and the ray will be moved to the character model that you want to convert. At this time, the ray will be displayed as a blue column. At this time, the user presses the relevant buttons on the handle to successfully complete the role's perspective exchange. Due to the sculpting problem of the size of some models, the characters need to be adjusted slightly after the exchange, so that the visitor's body and the outline of the character model are completely overlapped, and then the user experiences.

3. Optimal Implementation Steps for Virtual Reality Experience

As shown in FIG. 8, the user enters the data collection module of the system, and the data collection module is used for voice interaction with the user to obtain personal data such as the user's basic family situation;

1) Turn on the system and enter the setting module of the start unit

Set the number of characters who choose to be brought into the virtual world for sculpture experience and their physical height;

2) Choose your scene

Enter the scene setting unit

Scene options: long-distance picture display

Scene 1—wood grain floor
A white wall surrounded by white walls, without a ceiling, and a door in the middle of the wall
Sky, clouds
The sun is soft and abundant

Scene 2—Ocean Material Ground
Transparent wall on all sides
Sky at 18:00 in the evening
Sunset

Generated data: scene data

3) Create a role
The user selects the model of himself and the selected member from the models displayed in the model selection area
Specific options: model display
Gender: male, female
Basic body type: infant, juvenile, adult, elderly
Size adjustment: tall, short, fat, thin
Color adjustment: ten colors
Material adjustment: original wood grain material, low saturation color material
Generated data: character model data

4) Put your model into the scene

Checkerboard placement stage

Position: convert each character into a simple chess piece, and the scene into a chessboard grid, put the selected model in the grid, and record the position.

Orientation: After the character is placed in the grid, the user needs to choose the direction, there are 8 directions to choose from;

Generate data: position and orientation data of each character.

5) Generate your own virtual family grid

Input all the data stored in all previous steps as parameters into the virtual reality to generate a corresponding virtual reality world;

After the display is converted, a message appears saying please bring your virtual reality glasses for the next experience.

6) Start to experience

Bring virtual reality glasses; enter the virtual reality environment.

7) Simple operation learning

View angle switch: switch the handle button;

Move: limit the moving area, use the handle for long-distance displacement operation;

Grab: use the handle to grab and move the character model.

8) Scene roaming confirmation

First switch to the long-range perspective (third-person perspective) and observe in the established scene.

Is it satisfied (except position and orientation)?

Yes, go to step 9

No, go back to the previously unsatisfactory step (2,3) to readjust

9) First deep experience

According to the guidance of the consultant, the user enters the perspective of different characters through the handle selection.

First deep experience

Do I need to adjust the position of my family grid?

Yes, switch to the third person perspective, use the handle to grab and move the position and direction of the character model (the changed data needs to be recorded) and go to step 10

No, go to step 11

10) Second in-depth experience

Again according to the guidance of the consultant, the user uses the handle to choose to enter the perspective of different characters

Second in-depth experience

Are you satisfied?

Yes, go to step 11

No, switch to the third person perspective, use the handle to grab and move the position and direction of the character model (the changed data needs to be recorded here). Repeat step 10

11) Take a screenshot

Switch to third-person view roaming

Find the angle that suits you

Screenshot (save screenshot data)

12) The end

End module

Thanks the users for participating

Confidentiality, non-disclosure agreement is presented

It is recommended to record the mailbox number and collect data through the questionnaire mode.

The above-mentioned embodiments are only used to illustrate the technical ideas and features of the present invention, and their purpose is to enable those skilled in the art to understand the content of the present invention and implement them accordingly. This embodiment cannot be used to limit the scope of the present invention.

1. An immersive virtual reality interpersonal relationship sculpture psychological counseling assistive system, which is characterized in that it includes an interpersonal relationship sculpture construction module and a virtual reality experience module; wherein, the interpersonal relationship sculpture construction module is responsible for building a virtual reality interpersonal relationship sculpture, and the interpersonal relationship sculpture includes characters and scenes; the interpersonal relationship sculpture is a sculpture comprising characters and scenes formed by using a character model for the user's internal interpersonal relationship.

2. (canceled)

3. The system of claim 1 is characterized in that: wherein the virtual reality experience module includes a role interaction function module; the role interaction function module assists users to enter different target character models in the interpersonal relationship sculpture to implement role interaction; the target character model includes a user's own character model, an important person character model, or a mirrored character model; the mirrored character model is a bystander role outside the user's interpersonal relationship character model.

4-5. (canceled)

6. The system of claim 1 is characterized in that: the interpersonal relationship sculpture construction module comprises an interpersonal relationship construction basic function module and an interpersonal relationship construction interaction module; wherein, the interpersonal relationship sculpture construction basic function module comprises a character model library unit and a virtual scene model unit; wherein the interpersonal relationship sculpture construction interaction module is responsible for the user's selection and placement of character models and the formation of a virtual reality experience environment the character model library unit stores character models for expressing the user's internal interpersonal relationship; the internal interpersonal relationship of the user is a family relationship, a friend relationship, or a colleague relationship; the family relationship is a parent-child relationship, a husband and wife relationship, or a sibling relationship.

7-10. (canceled)

11. The system of claim 6 is characterized in that: the character model used to express the internal interpersonal relationship of the user is set according to the requirements of the character image; the character image requirements include gender, age, height, or fatness; the character model cannot only express the actual image of the user's internal interpersonal relationship object, but also can be used by the user to express the object's self-interpreted abstract image.

12-13. (canceled)

14. The system of claim 6 is characterized in that: the virtual scene model unit stores a scene model for users to place character models of themselves and others, and is responsible for constructing a virtual scene; the virtual scene model unit includes a time model and a basic space element model; the time model includes one or more of day, evening or night the basic space element model includes one or more of ground, sky, or wall.

15-17. (canceled)

18. The system of claim 14 is characterized in that: the virtual scene model unit includes a lighting unit or a material unit; the lighting unit is used to provide lighting design for the entire virtual scene; the material unit is used to implement material settings for the scene model and the character model; the virtual scene model unit includes a post-processing unit the post-processing unit is used to adjust and optimize the overall appearance of the scene to create a virtual environment that conforms to the user's psychological cognition.

19. (canceled)

20. The system of claim 6 is characterized in that: the interpersonal relationship sculpture construction interaction module comprises a virtual scene setting interaction unit and a character model placement interaction unit; the virtual scene setting interaction unit is used to provide a scene environment in which the character model is placed, including setting of time, space, lighting or model material; the character model placement interactive unit assists the user in completing the selection and placement of the character model; the character model placement interactive unit assists the user to complete the setting of the character model placement position, the character model placement distance or the character model orientation; the character model placement interactive unit includes the function of setting and highlighting the character's characteristics; the character characteristics include rights and status; the character model placement interaction unit includes one or more of a character model generation function module, a character model selection function module, a character model editing function module, or a data recording function module; the character model editing function module includes one or more of the character model rotation function module, the character model deletion function module, the character model movement function module, the character model operation cancellation function module, the character model color change function module or the character model height adjustment function module.

21-27. (canceled)

28. The system of claim 3 is characterized in that: the character model of the user is a real character model of the user or a character model of a character that the user imagines to be; the system includes a VR experience function module, which is used by the user to enter the completed interpersonal relationship sculpture for experience; the role interaction function module assists the user to complete the role interaction through the role interaction controller; the role interaction controller is a handle controller.

29-31. (canceled)

32. The system of claim 28 is characterized in that: the method for the role interaction function module to assist the user to enter the target character model through the role interaction controller includes the following steps:
(1) the user triggers the role interaction controller;
(2) the role interaction function module tracks the target character model through the role interaction controller, and the role interaction function module confirms the validity of the target character model and outputs the target character model information;
(3) the role interaction function module obtains the position and orientation of the target character model according to the output target character model information, and records the position data of the target character model;
(4) the role interaction function module obtains the user's position in the virtual space according to the position of the VR main helmet;
(5) role interaction: the role interaction function module sets the user's position as the position of the target character model, and the user presses the button on the character interaction controller to enter the target character model perspective;
(6) high degree of viewing angle processing and matching: to implement that the contours of the user's body and the target character model are completely overlapped.

33. The system of claim 32 is characterized in that: the character interaction controller emits a ray tracing target character model.

34. The system of claim 33 is characterized in that: the process of ray tracing the target character model is:
(1) the role interaction function module determines the starting position of the search ray according to the position and direction of the current role interaction controller, and starts the search ray tracing function; the role interaction controller emits a ray and moves the ray to the character model of the character to be interacted and selects the target character model;
(2) after confirming the validity of the target character model, the role interaction function module loads the target character model information and outputs the target character model information.

35. The system of claim 28 is characterized in that: the virtual reality experience module provides the handle controller to assist the user in switching perspectives in a virtual reality environment; the virtual reality experience module provides the handle controller to assist the user in grabbing or moving the character model in the virtual reality environment the virtual reality experience module provides the handle controller to assist the user to change the position or orientation of the character model remotely, thereby changing the interpersonal relationship sculpture space.

36-37. (canceled)

38. A psychological intervention method based on the immersive virtual reality interpersonal relationship sculpture psychological counselling assistive system is characterized in that it includes the following steps:
(1) the user enters the psychological consultation assistive system to set up the virtual scene of the interpersonal relationship sculpture;
(2) the user selects the character model of himself and others;
(3) the user places the character model in the virtual scene, and the system generates the user's interpersonal relationship sculpture;
(4) the user puts on the virtual reality device and enters the virtual reality environment;
(5) after the user enters the virtual reality environment, he confirms the scene;
(6) after the user confirms that he is satisfied with the current interpersonal relationship sculpture, under the guidance of the consultant, he can choose to enter the perspective of different character models through the handle controller for experience.

39. The psychological intervention method of claim 38 is characterized in that: in step 5, the scene confirmation includes: the user switches to the perspective view through the handle controller, and observes in the established interpersonal relationship sculpture; if the user is not satisfied with the current interpersonal relationship sculpture, the user can grasp or move the character model through the handle controller, change the position or direction of the character model, or return to step 1 or step 2, to adjust the virtual scene or character model settings.

40. The psychological intervention method of claim 39 is characterized in that: it includes step 7: after the experience is completed, if the user is satisfied with the experience, the experience is ended; if the user is not satisfied with the experience, the user can switch to the perspective view through the handle controller, use the handle controller to grab and move the character model and change the interpersonal relationship sculpture; the system records the position or orientation data of the character model after the change, and then the user again chooses to enter the perspective of different character models through the handle controller according to the guidance of the consultant to experience again.

41. The psychological intervention method of claim 39 is characterized in that: in step 2, the user selects a mirrored character model, which is a bystander character model outside the user's interpersonal relationship.

42. The psychological intervention method of claim 41 is characterized in that: the character model selected by the user in step 2 is the real character model of the user or the character model of the character that the user imagines to be.

43. The psychological intervention method of claim 42 is characterized in that: in step 6, the perspectives of different character models that the user chooses to enter include the user's own character model, the other person's character model, or the perspective of the mirrored character model; wherein the other person's character model is the character model of other members in the interpersonal relationship.

44. The psychological intervention method of claim 43 is characterized in that: the virtual scene setting in step 1 includes the setting of time, space or lighting.

45. The psychological intervention method of claim 44 is characterized in that: the character model selection in step 2 includes the choice of character model gender, age, height or fat or thin.
